(19) United States
(12) Patent Application Publication
(10) Pub. No.: US 2020/0173719 A1
(43) Pub. Date: Jun. 4, 2020

(54) Method and system for cold storage health and content monitoring

(71) Applicant: Mikko Lauri Antti Jaakkola, San Diego, CA (US)

(72) Inventor: Mikko Lauri Antti Jaakkola, San Diego, CA (US)

(21) Appl. No.: 16/208,503

(22) Filed: Dec. 3, 2018

(51) Int. Cl. F25D 29/00; G01M 13/00; G01N 33/00; G08B 21/18

(52) U.S. Cl. CPC F25D 29/008; G01M 13/00; G01N 33/0047; G08B 21/182; G01N 33/0054; G01N 33/0063; G01N 33/004

(57) ABSTRACT

A method and device for monitor health and content of cold storage units are disclosed herein. In the embodiments, one or more cold storage health and content monitor devices 100 per cold storage unit are connected into an existing power- and communication-infrastructure communicating with a server computer system 110 that communicates with applications 120 sending notifications and receiving their inputs. The monitoring device includes capacitor energy storage for wide operation range and fast charging as well as plurality of sensors. Further, for some embodiments, the device may further comprise LED lights, a speaker and one or more microphones. The notifications are send when at least one of the previously set conditions meet the threshold. These conditions include, but are not limited to, monitoring cold storage unit equipment, monitoring content changes and detecting harmful biological processes inside the unit. The notifications are delivered through natural voice interface and using digital communications.

### TECHNICAL FIELD

The present disclosure relates monitoring cold storage units such as freezer and refrigerators through gas-, temperature, humidity- and air-pressure-sensors, RFID-tags, accelerometer, microphones and (super-)capacitors by drawing the power and offering functionalities such as emitting light and audio user-interface.

### BACKGROUND—PRIOR ART

The following is tabulation of some prior art that presently appears relevant:

### U.S. PATENTS

| Patent Number | Kind Code | Issue Date | Patentee |
|---|---|---|---|
| 9,661,713 | B2 | May 23, 2017 | Sandhiprakash J. Bhide |
| 9,500,532 | B2 | Nov. 22, 2016 | Harry J. Schechter |
| 9,373,164 | B2 | Jun. 21, 2016 | Kyehwan Lee |
| 7,716,935 | B2 | Sep. 1, 2009 | Yong Tae Kim |
| 9,170,047 | B2 | Oct. 27, 2015 | Nam Ju PARK |

### U.S. PATENT APPLICATION PUBLICATIONS

| Patent Number | Kind Code | Issue Date | Patentee |
|---|---|---|---|
| 2015/0267960 | A1 | Sep. 24, 2015 | Sandhiprakash J. Bhide, |
| 2014/0118135 | A1 | May 1, 2014 | Michael O'Brien |

### NONPATENT LITERATURE DOCUMENTS

https://shop.sensorpush.com/, downloaded Nov. 5, 2018.
https://www.ethylenecontrol.com/products/filtration-system/, downloaded Nov. 5, 2018.

### BACKGROUND

The background description provided herein is for the purpose of presenting the context of the disclosure. Unless otherwise indicated herein, the materials described in this section are not prior art to the claims in this application and are not admitted to be prior art by inclusion in this section.

People disposed 37.6 million tons of food waste in United States in 2015. Good amount of the waste is due to improper storage conditions, running out of the natural expiration dates, and an equipment malfunctions. Food poisoning is also a major issue as 48 million people get sick, 128,000 are hospitalized, and 3,000 die from foodborne disease in USA each year.

Refrigerators, refrigerator rooms, freezers hereon referred as cold storage units intent to lengthen the applicable usage time for the stored goods. This is achieved by lowering temperature to reduce the rate of the metabolic and other changes in the cells or chemicals as well as slowing the growth of bacteria and fungus in organic materials. However, equipment failures, incorrect storing configuration and storing practices all lead to accelerated decomposition and expiration of the goods. This process can be relatively quick as Foodsafety.gov recommends to discard any perishable food (such as meat, poultry, fish, eggs, and leftovers) that have been above 4° C. (40° F.) for over 2 hours making early detection vital to manage potential damages.

It is not just the owners and users of the cold storage units who are interested learning possible equipment failures as soon as possible but there is also an insurance industry that is offering content coverage for the cold storage units like freezers. Any innovations that can prevent and reduce the amount of damage has great value for such operators.

Most cold storage units currently include internal thermometer exposed to the user but they are often placed inside the cold storage unit providing no visibility without opening the unit door. There are also products like SensorPush Wireless enabling cold storage unit temperature and humidity monitoring outside the device but these types of solutions operate in close proximity to the display unit or require dedicated infrastructure. Also having internal battery reduces the long-term active users as people tend to forget to charge and consider such activity as a nuisance.

Commercial deployments sometimes have remote monitoring systems with plurality of sensors and they are fixed installations with either wired or wireless Internet support. These solutions are typically focused on monitoring coarse equipment and climate parameters requiring dedicated installation. Some commercial solutions focus on removing ethylene that is ripening gas generated by the plants. These solutions slow the process of ripening down but cannot reverse the process. Eventually spoiled food will cause bad smell turning some foods into liquid mass of bacteria and biomass.

Another types of embodiments of cold storage unit content monitoring system are the ones built directly into the unit. There are newer generation cold storage units with Internet connectivity and advanced airflow systems often including filtering and shelf specific temperature control that helps to slow some of the metabolic decomposition process, but those features are limited to the higher-end models and do not solve aftermarket uses. The typical lifetime of a cold storage unit is between 14 and 17 years, according to “The Expected Life of a Refrigerator” web-page, implying that massive existing installment base will not get these features anytime soon.

### SUMMARY

The disclosure is cold storage unit health and content monitoring device and method with Internet connectivity having different embodiments optimized for various existing cold storage power infrastructures while using plurality of different types of sensors and performing fusion with them to construct a complete view of the cold storage unit status, its content, possible risks facing the unit and notifying the subscribing party on predetermined conditions. The device stores energy for its operations in a capacitor. The capacitor is advantageously connected to a power supply present in the cold storage unit, such as the light bulb base.

The cold storage can take a form of a refrigerator, a freezer, a walk-in cooler, a walk-in freezer, a refrigerated container, a refrigerated trailer, and any storage meant for controlling its climate.

### Advantages

Accordingly several advantages of one or more aspects are as follows: providing maintenance free aftermarket health and content monitoring solution enabling the users to maximize the value of their existing cold storage unit that provides remote interactions and alarms, that provides novel means to detect any abnormalities in compressor operations to provide the owner a longer time window to prevent complete shutdown, that enables timely voice interactions with natural language, that can detect and warn about abnormal environmental conditions such as spoiled food and other decomposition related hazards, that detects door sealing issue, that helps with the cooling unit inventory management, that works as the last line of defense as burglar alarm. Other advantages of one or more aspects will be apparent from a consideration of the drawings and ensuing description.

### DRAWINGS—FIGURES

In the drawings, closely related figures have the same number but different alphabetic suffixes.

FIG. 1 is block-diagram of the overall cold storage health and monitoring system.

FIG. 2 is refrigerator light bulb replacing embodiment of the cold storage health system and monitoring device.

FIGS. 3A and 3B are wall-socket and wireless power transfer embodiments while FIG. 3C is the top view shared by the both embodiments.

FIG. 4 is system diagram of cold storage health and monitoring device, in accordance with various embodiments.

FIG. 5 illustrates how capacitor voltage is changed when charged from zero and then discharged.

FIG. 6 is an example of the state-diagram mapping sensor activation to the system states.

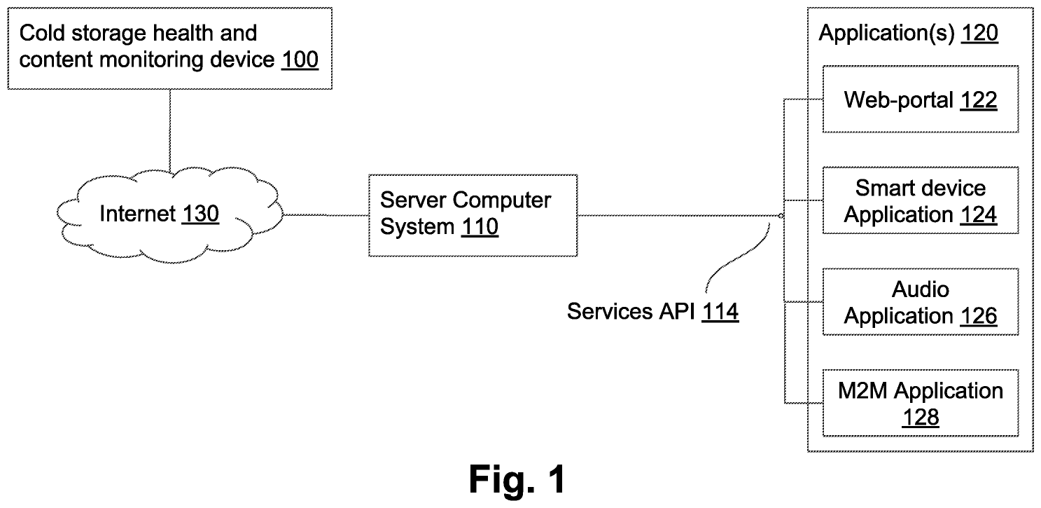

### DETAILED DESCRIPTION—FIGS. 1, 2, 4, 6—FIRST EMBODIMENT

The high-level operational environment for the closure is illustrated in FIG. 1. There Cold storage health and content device 100 is connected to Server Computer System 110 through Internet 130. Server Computer System 110 may have one or more Applications 120 serving them through its Service API 114.

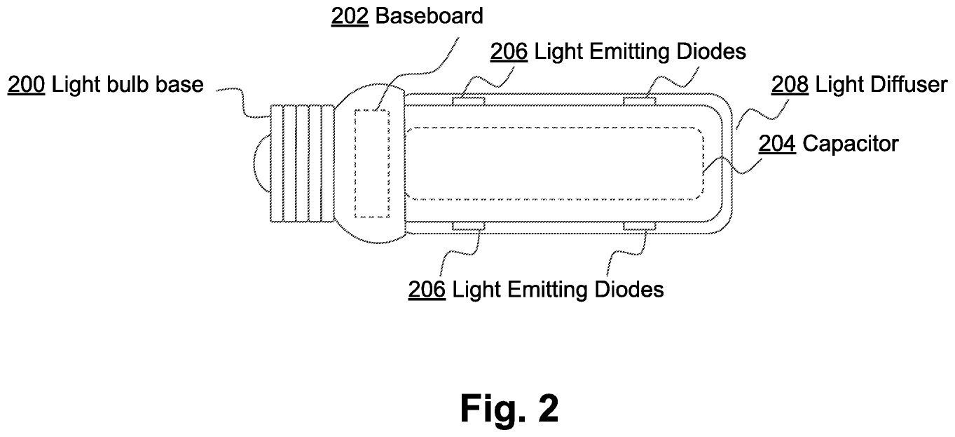

One embodiment of the closure is illustrated in FIG. 2. The embodiment has light bulb base 200 to enable the attachment to the existing cold storage light bulb receptor to provide external power. The embodiment uses (super)-capacitor as the energy storage and is connected to baseboard 202. The embodiment may include Light Emitting Diodes (LED) 206 as it replaces the existing light bulb to avoid removing an existing functionality. If LEDs are part of the embodiment construct, it also includes light diffuser to spread the light. Baseboard 202 is included to the base of the embodiment in FIG. 2. but can reside anywhere in the embodiment and, in some implementations, could be split into multiple parts that are connected.

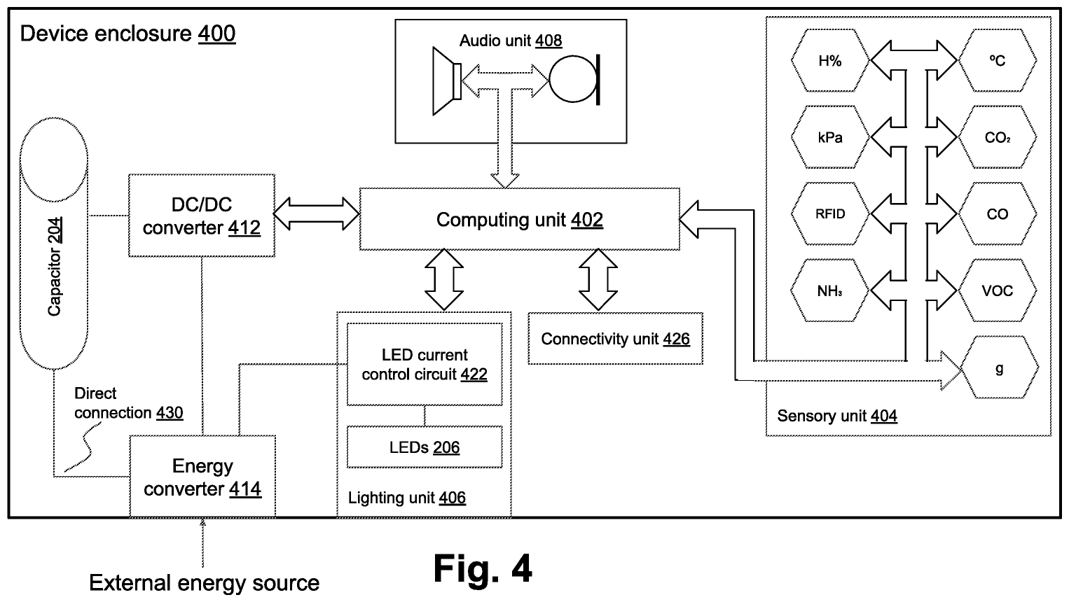

The overall system architecture of an embodiment of the invention is illustrated in FIG. 4. Baseboard 202 includes:
a. Computing unit 402
b. DC/DC (Direct Current to Direct Current) converter 412
c. Energy converter 414
d. Connectivity unit 426
e. Sensory unit 404
f. Audio unit 408

Computing unit 402 includes System-on-chip (SoC) that provides a processor and logic (in the form of IP-blocks) for controlling peripheral devices as well as memory to store data and execute the program code.

In this embodiment Energy converter 414 takes a form of AC/DC (Alternative Current to Direct Current) converter and it is connected to a DC/DC converter and, depending on the component selection, could also connect directly into the capacitor 204 as illustrated with connection 430.

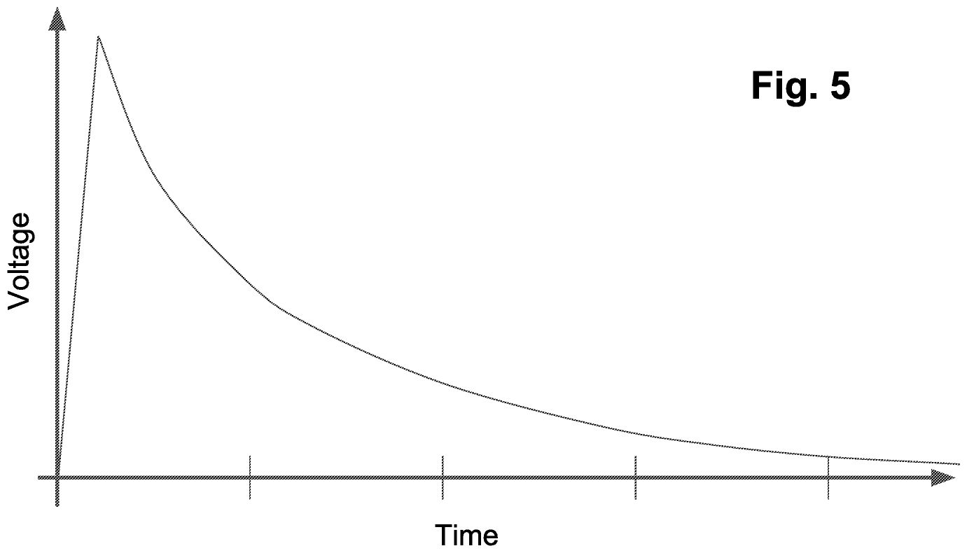

DC/DC converter 412 is powering computing unit and, depending on the circuit board design, could also directly do so to the other subsystems. When the energy converter 414 is getting outside power, it connects to DC/DC converter that feeds the adjusted voltage to the other systems and charges the capacitor. When the energy converter 414 is off, DC/DC converter is powered by the capacitor. DC/DC converter keeps the operating voltage regulated as when capacitor is discharged its voltage drops in linear fashion, as illustrated in FIG. 5, and the other components, such as computing unit, in the system require constant voltage. Using capacitor as the energy source is one of the novel aspects of the invention compared to the prior art that use batteries. Cold storage unit door is only open for a brief moment typically lasting between 5-30 seconds, so all the energy is stored in short time-frame making the charging power high compared to the discharge requirements. Capacitors support large recharge cycles and high charging power, that are typical in the given application, making capacitors ideal given their operating parameters compared to the traditional chemical-based solutions such as lithium-ion batteries. Also low temperatures varying from −30° C. (−22° F.) to +5° C. (41° F.) would make it difficult to use traditional chemical batteries while capacitors are capable operating in temperatures as low as −40° C. (−40° F.) without significantly losing the storage capacity and charging speed. Capacitor could be either form of traditional capacitor or supercapacitor depending on how much and fast the energy needs to be stored in the given embodiments of the disclosures.

Connectivity unit 404 enables communication between computing unit 402 and Server computer system 110 illustrated in FIG. 1. The disclosure may use Internet Protocols for its information exchange between the nodes. The connectivity unit may include communication interface, such as LTE (Long Term Evolution), Wifi, Bluetooth, Z-wave, ZigBee, LoRa, NB-LTE (Narrow-band LTE).

Sensor unit 404 may include (without limitation) one or more of following sensors:
Accelerometer (g)
Volatile Organic Compound (VOC) gas sensor
Ammonia (NH₃) gas sensor
Carbon Monoxide (CO) gas sensor
Carbon Dioxide (CO₂) gas sensor
Humidity sensor (H₂O %)
Temperature sensor (° C.)
Air pressure sensor (kPa)
RF ID tag-reader

These sensors are connected to the computing unit that controls their operation and collects the sensor data at predetermined times. VOC sensor can alternatively be replaced with any combination of individual ammonia, ethylene, methane, hydrogen sulfide sensors.

The closure may also include Audio unit 408 comprising one or more speakers and microphones that are connected to computing unit 402. One microphone is sufficient for most audio capture use cases but adding more microphones enables beamforming helping to isolate audio sources and filter unwelcomed noises.

The closure may also include Lighting unit 406. This enables retaining the cold storage unit functionality the same when the closure is replacing the existing cold storage light bulb. As a typical refrigerator are designed for incandescent lamp using all the drawn power into generating light, the maximum specified light bulb specification implicitly makes that the maximum power budget for the embodiment. Using lower power light sources such as LEDs helps to reserve the saved power budget for charging and running its other subsystems. Typical 40 W incandescent light bulb can be replaced with 9 W worth of LEDs giving sufficient amount of energy for the embodiment. Lighting unit 406 comprises LED current control circuit 422 that's communicably coupled to the computing unit and electrically connected to one or more LEDs, and Energy converter 414 or the computing unit.

### Operations—FIGS. 1, 2, 4, 6

Cold storage health and content monitoring operates by having cold storage health and content monitoring device 100, hereon called as the embodiment, to send data over Internet to Server Computer System 110 that performs further processing to the data and delivers notifications through its Services API 114 to Applications 120. There are different types of applications that can subscribe to the Service API to get notifications from the Server Computer System. Recipients can subscribe for notifications such as application or web-page notification, emails, text-messages, audio messages, robocalls, and Internet chat application-message such as Messenger-messages. Audio messages and notifications can also be delivered directly through the embodiment speaker(s). Services Computer System 110 can also initiate the communication towards the embodiment to configure its parameters, upload new computer software and audio messages.

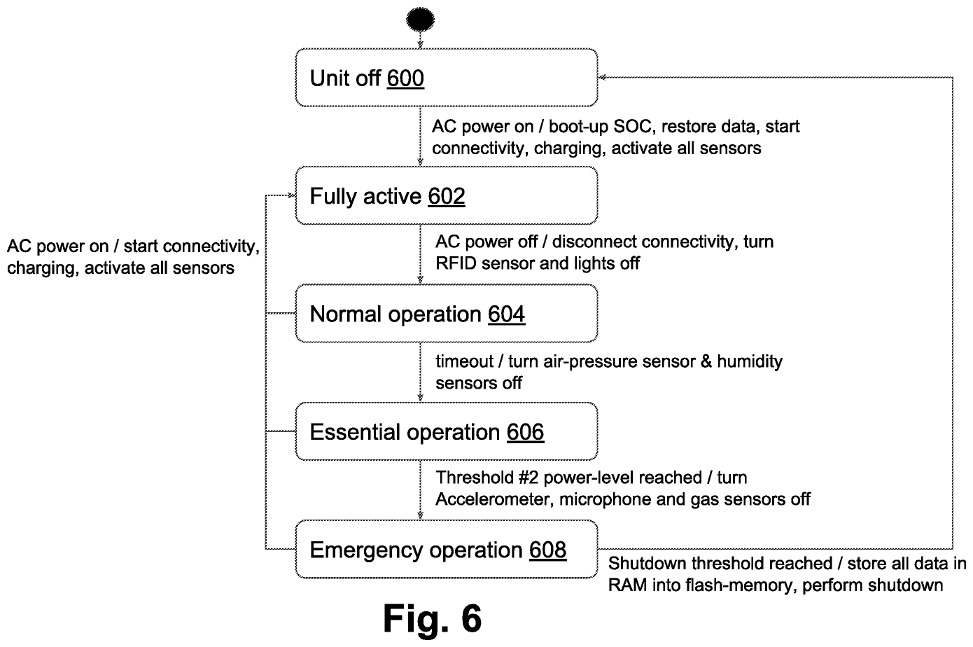

When the cold storage unit door is opened and internal light bulb receptor is powered-up, the embodiment starts charging the capacitor and signals the external power use through DC/DC converter to the computing unit that changes the system state into Fully active 602 as illustrated in FIG. 6. The computing unit activates all the sensors, establishes the connection to the gateway through the connectivity unit and commands the Lighting unit to turn LEDs on. Once sensors are active, the computing unit measures after predetermined delay their values, sends both, the just captured and previously stored, sensor measurement data to Computer Server System 100. The computing unit also scans using RFID reader the content information of the cold storage unit as long as the door is open. This is also the time, when the audio units both listens the audio input, delivers any pending notifications to the applications and the user opening the cold storage door. If the cold storage unit power is on longer than the predetermined time, the system creates “door open”-notification to the applications to help the user to check if the door has been accidentally left open by accident. The embodiment can also be configured through Service API 114 to provide notification each time when the cold storage unit is opened.

The disclosure uses RFID technology to detect the content of the cold storage unit. RFID technology has been existing in various forms around 20 years but due to its cost, it hasn't spread outside payment and specialized RFID tags. However, the lowered cost of the technology and introduction of cashierless stores will expand its use in the near future. Detecting content changes is ideal when the cold storage unit door is open or when just being closed as after that, the content won't change. Also, continuous RFID scan uses energy in both RFID receiver and computing unit, so it is done when the embodiment is having the external power. Content management can serve many purposes in the cold storage unit such as sending notifications when noticing that some items have gone below predetermined threshold to help the owner ensure sufficient supply of the needed goods. Being aware of the cold storage unit content optimizes the use of the information received from other sensors. For example, knowing the cold storage internal gas composition and the stored items can enable more accurate identification, for example, the decomposing or expired food item. Also, some foods are better preserved in different humidity levels and temperature so making the cold storage unit user aware of the optimal conditions can help to extend their life.

When the energy converter 414 loses its external power, the computing unit transitions into Normal operations 604 state turning lights, RFID sensor and connectivity unit off or into low-power mode.

The embodiment measures air pressure using an air pressure sensor to detect the quality of the seal between the cold storage unit door and its frame. When the cold storage unit door is opened, the unit is filled with surrounding warmer air. Once the door is closed, the air temperature inside the unit starts to drop and the pressure follows Combined gas law or generally known as General Gas Equation.

The formula states that as the volume and the amount of material, air in this case, stays constant while temperature gets lower, the pressure must get lower too. In this case, it creates minor vacuum due to the air pressure difference between outside and inside the cold storage and thus enables detecting if there is a good seal. Notification is sent if poor sealing or door being ajar is detected. Good seal is important to help saving energy, reduce wear and tear, and ensuring the optimal storage conditions.

Humidity is another cold storage climate parameter to monitor and control. Excess humidity has adverse effects and can be a sign of equipment malfunction. Humidity in freezer will cause ice buildup and can be an indication for leaking ice dispenser. In refrigerator, excessive humidity can be a sign of a decomposition but also on its on it will cause foods to rotten faster as well as lead into mold build-ups. Humidity sensor provides the data through Service API 114 to the applications 120 that have subscribed for the notification enabling the interested party to take an action to address the abnormalities. Humidity is typically not having major fluctuation in short-period of times after the door is closed and the same applies to air pressure sensor after the quality of seal has been detected so both sensors will be turned off or placed into lower power mode when the embodiment moves into Essential operation 606 state.

Temperature has significant impact on the speed of chemical and biological processes. For example, food stored in the wrong temperature will be adversely impacted either by damaging taste if being too cold or making the food to spoil faster in case the temperature is too high. All cold storage units have one or more temperature sensors to control the temperature and operations of the compressor, but it is often impossible or inconvenient for the user to see the target temperature and how it is being followed without opening the unit and manually consistently recording the values.

Many current cold storage units will provide warning sound or light based indicator if the temperature becomes too high but this only happens in the cases where there are significant deviations to the target values that make it difficult for the user to identify those cases when the temperature increases are consistently elevated but have not reached the threshold levels while still causing negative impact to the unit content. The alarm indicating the change of the temperature beyond operational limits is typically only useful if somebody is in close proximity to the cooling unit and providing the alarm over Internet solves this issue. Temperature warning over Internet is also useful in the cases where the cooling unit is having major malfunction, for example, in the case of power outage or broken compressor. As the embodiment is also recording the content of the unit, it can match the temperature information against food spoilage recommendations in the case of malfunction to notify on those food items that are recommended to be thrown out in order to avoid, for example, food poisoning and also advice on those items that are still safe to use to help save money and wasting food.

Monitoring compressor operations is performed using a microphone and accelerometer as well as using the change in temperature sensor during the operation. In most cold storage implementations, accelerometer can detect change in acceleration when the compressor is starting and stopping. Accelerator typically reads close to zero value when the compressor is off but when it is turned on and off there is going to be a change in acceleration signaling the start or stop times. Alternatively, the microphone can listen surrounding environment and determine if the sound heard belongs to the compressor and that way determined the starting and stop. As both using a accelerometer and a microphone requires filtering to detect false positives, there is an option to use both methods in combination to get the best possible accuracy. Duration of the compressor operation can be calculated by capturing the start- and stop-time. As the compressor is running at the constant power and duration is known, the unit energy consumption can be calculated using formula: E=Pt if the power-rating has been inserted through an application.

Sound can be used beyond determining start- and stop-time of the compressor. The sound sample of the compressor operation can be processed through frequency domain analysis to detect changes in the operating frequency composition, amplitude and phases where changes over longer period of time can be indicative for underlying issue that warrants sending a warning notification. Also, compressor operation duration per temperature change provides indication of the operational efficiency of the compressor and if the predetermined threshold is reached, a notification is sent, for example, to highlight that it is time to do dusting and clean the compressor coils. Having knowledge of the compressor operations can also be used to provide early notifications of the equipment malfunction. Over time the embodiment learns the trigger temperature for the cold storage unit when the compressor typically is turned on and if this doesn't happen, the operator of the unit can be notified immediately as it is a sign of unexpected behaviour instead of waiting major changes in the temperature. This method has significant advantage over the prior art as temperature-only based warnings cannot ever be made as sensitive as they need to tolerate normal external imposed temperature fluctuations such as people inserting warm food into the cold storage unit. Adding warm food into the unit will increase the inside temperature but as long as the compressor has been started, the condition is temporary, and the unit works as specified.

Advanced gas sensors, as found in the embodiment, can help to protect the cold storage unit content through early detection of excessive decomposition and ripening in the unit. It is not uncommon to find something buried at the bottom of the storage containers that is in advanced phases of decomposing and partly turned into a liquid called leachate. Situations like this are not just unpleasant to clean but also accelerate the ripening and rotting the rest of the exposed foods in the unit leading significantly shorter shelf-life and thus causing vicious cycle where rotting leads into more rotting. Decomposition also releases energy forcing the cold storage unit compressor to do extra work retain the specified temperature that is then visible in the energy consumption.

There are two kind of decomposition reactions; aerobic and anaerobic. Aerobic decomposition uses oxygen as part of the breaking down with the organic material producing water, carbon dioxide and energy while anaerobic process uses water with the organic matter producing methane, hydrogen sulfide (rotten egg smell), and energy. Another relevant gas is ethylene that serves as a hormone in plants regulating the ripening of the fruit. Plants produce the gas naturally during certain stages of growth or if the plant have been damaged. Ethylene gas doesn't just act as an indicator about the ripening and/or rotting of an individual fruit, but higher ethylene gas levels make the other plans to ripen faster creating chain-reaction. This is the reason why one rotten fruit can easily lead faster rotting of all other fruits and vegetables in the cold storage unit. Ancient Egyptians used this technique to accelerate enhance ripening by damaging plants. Ethylene generators are used in commercial environments for similar purposes to shorten ripening process today. In use cases where the aim is to extend shelf life, the amount of ethylene needs be controlled inside the storage unit. Ammonia is another gas to monitor as its presence is an indication of meat decay. By monitoring the individual and total concentrations of methane, carbon dioxide, humidity ethylene, ammonia and hydrogen sulfide, it is possible to conclude the overall condition of the cold storage unit stored goods and send notification when predetermined threshold levels have been reached. Humidity and the mentioned gases can be measured using combination gas sensors such as Volatile Organic Compound (VOC) sensor or dedicated sensors for subset or the individual gasses.

Once the embodiment reaches emergency power level, it shutdowns or moves into low-power mode, for example, all but temperature sensor and moves into Emergency operation 608 state that still enables it to notify the temperature based issues. When the embodiment determines that shutdown is inevitable, it sends the latest data to Computer Server System 110, stores its working memory (RAM), performs other clean-up operations to ensure as fast reboot as possible once AC power is active again, and powers the system completely off.

Even though the system follows FIG. 6 sensor power states, any of the sensors can still be periodically read to track the cold storage unit state in all but the complete shutdown.

The operations of the system can be optimized by providing additional information through Applications 120. Information about the size, type, make and model of the cold storage unit can be provided through the applications to calibrate the system parameters. For example, the growth in ethylene gas concentration due to rotting broccoli in walk-in cold storage is much slower due to higher volume of air than in small refrigerator. Having this information helps settings the cold unit storage optimized notification thresholds.

The application may also configure the system into “holiday-mode” where system aggressively moves through the states in FIG. 6 to extend the energy storage to last as long as possible. The system can also be set to send notifications if the door has been open for certain threshold value. This can be useful for teaching children for proper cold storage behaviour by giving audio notification when the door has been open for too long. The system can also be configured into security monitor state, where it listens all outside noise and monitors door operations, and sends alarm if the monitored events have been detected.

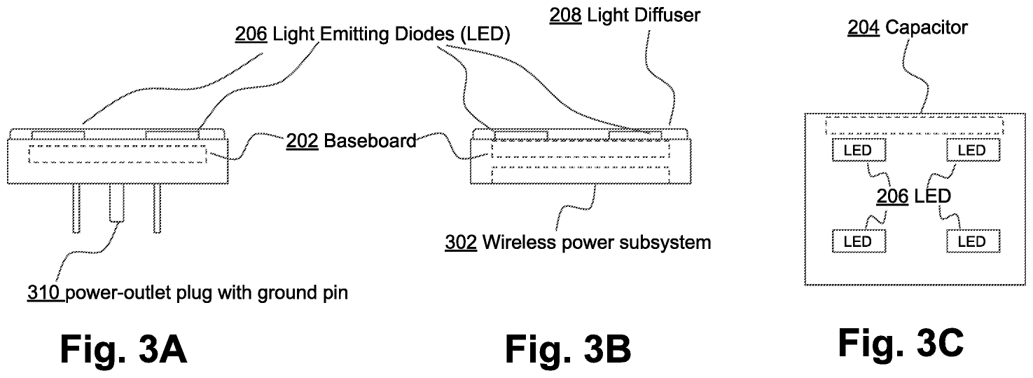

### FIG. 3—Some Alternative Embodiments

Two alternative embodiments are shows in FIGS. 3A, 3B and 3C. The top view FIG. 3C is shared by the both embodiments. These embodiments can optionally support lighting unit as shown in FIG. 3C to offer extra light to the cold storage unit. As the embodiments are not replacing any existing infrastructure, the form-factor doesn't need to follow any particular form-factor, like light bulb, and in the figures square flat design has been illustrated to optimize the embodiment space requirements.

The embodiment illustrated in FIG. 3B is using wireless power transfer technology enabling greater levels of freedom for its placement inside the cold storage unit while the design in FIG. 3A uses an existing power-outlet. Power-plug 310 is just one of the means for acquiring wired power and it can also take a form of USB- and Power-over-Ethernet.

The embodiment in FIG. 3B is using the same connectivity unit 426 interfaces as the embodiment in FIG. 2 but the embodiment in FIG. 3A can also include wired connectivity technologies such as Ethernet, IP over USB, and Power-line communication interfaces.

Both alternative embodiments can also include capacitor 204 as in the embodiment in FIG. 2 but as there is more constant supply, there is an opportunity for downsizing.

### CONCLUSIONS, RAMIFICATONS, AND SCOPE

Accordingly, the reader will see that cold storage unit health and content system with its disclosed embodiments offer carefully optimized novel maintenance free low-cost solutions for cold storage units without requiring any new infrastructure or removing existing functions.

In addition, having fusion over plurality of the sensor enables creating more complete picture of the status of cold storage unit, provide timely, and actionable information to the user.

Furthermore, the disclosure has the additional advantages in that:
1. Natural interaction through voice interface for the users at the optimal context.
2. Offers cold storage unit content detection enabling various use cases from estimating expiration dates to overall inventory management.
3. Enables users to save in their food bill by detecting decomposition processes and advising the user to remove content that accelerates the decomposition of the other content
4. Helps saving in energy bill by giving the users the tools to optimize the cold storage unit climate, identifying when the unit requires maintenance or cleaning, and by identifying energy producing decomposition processes

Although the description above contains many specificities, these should not be construed as limiting the scope of the embodiments but as merely providing illustrations of some of several embodiments. For example, one could create an embodiment for natural wine cellar without microphone, RFID, accelerometer and air pressure sensors as there is no compressor to monitor, open space where no pressure difference is being expected and voice functionality is seen as irrelevant function while any of the disclosed external power options might work. Alternatively, another embodiment without RFID and lighting unit would work as low cost remote sensor system for homemade greenhouses.

Thus, the scope of the embodiments should be determined by the appended claims and their legal equivalents, rather than by the examples given.

1. A cold storage health and content monitoring device, comprising:
a sensory unit configured to monitor the cold storage unit state and content;
a connectivity unit sending notifications to applications;
a capacitor for energy storage enabling monitoring, notifications and communication with a server computer system when cold storage unit is not providing energy to said device;
a computing unit electrically and communicably coupled to said capacitor, said sensory unit and, said connectivity unit; and
wherein said computing unit operates said sensory unit to monitor cold storage state and content changes, and communicates with a server computer system via said connectivity unit and the device is adapted to draw power from said capacitor.

2. The cold storage health and content monitoring device of claim 1, wherein the cold storage is one of a refrigerator, a freezer, a walk-in cooler, a walk-in freezer, a refrigerated container, a refrigerated trailer and any storage benefiting of health and content monitoring to help to preserve its content.

3. The cold storage health and content monitoring device of claim 1, wherein said sensory unit includes at least one of a temperature sensor, an air pressure sensor, a humidity sensor, an accelerometer sensor, a volatile organic compound gas sensor, an ethylene sensor, a methane sensor, a hydrogen sulfide sensor, a carbon monoxide sensor, a carbon dioxide sensor, and RFID reader.

4. The cold storage health and content monitoring device of claim 1, further comprising a DC/DC converter electrically coupled with said capacitor;
an energy converter electrically coupled with said DC/DC converter and said capacitor; wherein said computing unit detects power state changes and is electrically and communicably coupled to said capacitor via said DC/DC converter, and the device is adapted to draw power through the energy converter.

5. The cold storage health and content monitoring device of claim 4, wherein said energy converter comprises at least one of an AC/DC power converter, a Power-over-Ethernet power converter, a wireless power transfer power converter, and a DC power converter, and wherein a physical power connector is consist one of a light bulb male connector, a power outlet male connector, a Ethernet-port, an antenna for wireless power transfer technology, and a USB-connector.

6. The cold storage health and content monitoring device of claim 1, wherein the device further comprises one or more speakers, and one or more microphones that are coupled to said computing unit.

7. The cold storage health and content monitoring device of claim 1, wherein said connectivity unit comprises at least one of LTE, Wifi, Bluetooth, Z-wave, ZigBee, LoRa, NB-LTE, Ethernet, and Power-line communication interfaces.

8. The cold storage health and content monitoring device of claim 1, wherein said capacitor is one of an electrolytic capacitor, a super-capacitor, a double-layer capacitor, a pseudo capacitor, and hybrid capacitors of different classes.

9. The cold storage health and content monitoring device of claim 1, wherein the system further comprising:
a LED current control circuit communicably coupled to said computing unit and electrically coupled to said computing unit or said energy converter; plurality of light emitting diodes (LED) electrically coupled to said LED current control circuit; and
Light diffuser overlaid on top of LEDs and attached to the body of the embodiment.

10. A method for monitoring cold storage health and content and notifying on changes with a device comprising:
Monitoring cold storage state and content changes using a sensory unit and a computing unit;
detecting a change in the state of the cold storage;
notifying on the state changes; and
using a connectivity unit to communicate with a server computer system.

11. The method for monitoring cold storage unit health and content of claim 10, wherein the state of the cold storage unit comprises at least one of open cold storage door ajar cold storage door, and improper sealing between the door and the gasket, and the detecting comprises measuring air pressure changes inside the cold storage unit using said sensory unit and computing unit powered by at least one of said capacitor and energy converter.

12. The method for monitoring cold storage unit health and content of claim 10, wherein the step of detecting comprises detecting content changes inside said cold storage unit includes detecting the predetermined items that have depleted, using content information to determine the optimal environmental settings for the stored items, using content information to determine the items that should be discarded and can be saved in the case the predetermined content storage climate criterium have been exceeded, and notifying these changes to the interested parties using said sensory unit, said computing unit and said connectivity unit powered by at least one of capacitor and energy converter.

13. The method for monitoring cold storage unit health and content of claim 10, wherein the step of detecting comprises monitoring cooling unit compressor operations, including capturing at least one of compressor sound, start and stop times, duration and cold storage vibration levels of said compressor operations and further comprises at least one of calculating temperature change, estimated energy-consumption, early malfunction detection and overall compressor condition estimate using said sensor unit and computing unit powered by at least one of said capacitor and energy converter.

14. The method for monitoring cold storage unit health and content of claim 10, wherein said the step of monitoring comprises measuring cold storage unit content ripening and decomposition processes are detected by monitoring changes in concentration and levels of at least one of volatile organic compound gases, ethylene, methane, hydrogen sulfide, carbon monoxide, carbon dioxide, ammonia, and humidity using said sensory unit and computing unit powered by at least one of said capacitor and energy converter.

15. The method for monitoring cold storage unit health and content of claim 10, wherein the step of notifying comprises sending a notification taking the form of at least one of the following categories: an application notification, a web-page notification, an email, a text-message, an audio message, robocall, and Internet chat application-message using said computing unit and connectivity unit.

16. The method for monitoring cold storage unit health and content of claim 10, wherein said sensory unit comprises one or more microphones and the step of notifying comprises use of one of more speakers providing audio notifications, including voice, of detected predetermined conditions and audio input functionality to support natural language input processing using said computing unit and connectivity unit powered by at least one of said capacitor and energy converter.

17. (canceled)

18. The method for monitoring cold storage health and content of claim 10, wherein the method further comprising of communicating said cold storage unit sensor data with a server computer system using said computing unit to consolidate the inputs from all said sensory unit and sending notifications to applications if a condition is meeting at least one of said state change conditions that said applications have subscribed to.

19. The method for monitoring cold storage health and content of claim 18, wherein said application is at least one of a voice application operated in said connectivity unit, a smart device application, a Machine-to-Machine application, and a web-portal.

20. The method for monitoring cold storage unit health and content of claim 18, wherein the method further comprises a step of at least one application subscribing for a set of notifications for at least one cold storage unit, and configuring the at least one cold storage unit to send the set of notifications the at least one application has subscribed to the at least one application.
